(19) United States
(12) Patent Application Publication
(10) Pub. No.: US 2009/0118601 A1
(43) Pub. Date: May 7, 2009

(54) IR SPECTROGRAPHIC APPARATUS AND METHOD FOR DIAGNOSIS OF DISEASE

(75) Inventors: John F. Rabolt, Greenville, DE (US); Mei-Wei Tsao, Wilmington, DE (US)

Correspondence Address: RATNERPRESTIA, P.O. BOX 1596, WILMINGTON, DE, 19899, US

(73) Assignees: UNIVERSITY OF DELAWARE, Newark, DE (US); MATERIALS RESEARCH SERVICES, Wilmington, DE (US)

(21) Appl. No.: 11/576,229

(22) PCT Filed: Sep. 29, 2005
(86) PCT No.: PCT/US2005/034903

§ 371 Date: Jan. 15, 2009

Related U.S. Application Data
(60) Provisional application No. 60/613,759, filed on Sep. 29, 2004.

(51) Int. Cl. A61B 5/1455

(52) U.S. Cl. 600/318; 600/475

(57) ABSTRACT

A method for detecting disease in a patient includes providing infrared (IR) light and coupling the IR light through direct lens coupling or through a first group of one or more optical fibers. IR light is reflected from a portion of the patient and collected by a lens arrangement or a second group of one or more optical fibers. The reflected IR light is dispersed into its spectrum which is detected and analyzed. An apparatus suitable for diagnosing a disease in a patient includes an IR light source and optical fiber or direct lens coupling of IR light onto a body part or fluid of the patient. Reflected light from the patient is optically dispersed using a prism or grating. An IR focal plane array receives the optically dispersed light. The spectrum of the reflected IR light is used to provide a diagnosis of disease in the patient by identifying various disease markers or chemical fingerprints. The method and apparatus are capable of non-invasively detecting disease markers in a patient.

This application claims priority to U.S. Provisional Patent Application 60/613,759 filed on Sep. 29, 2004, the entire contents of which are incorporated herein by reference.

### BACKGROUND

This disclosure is directed to an IR spectroscopic apparatus and method for diagnosing disease, and is particularly related to a planar array infrared (PAIR) method and apparatus.

The advanced detection of disease is the goal of numerous global research initiatives into noninvasive in vivo methods of characterization. Many of these efforts focus on non-specific detection of the early manifestations of disease (e.g., cataracts in the eye, glaucoma, etc.), while others are designed for disease prevention, to check for the presence or absence of a specific chemical component (e.g., progesterone in saliva) in the body, i.e., a “disease marker” or “fingerprint”.

For many years the primary examination used to detect the beginning of cataracts has involved the dilation of the pupil so that light can enter the lens and be focused on the retina. A visible inspection by an ophthalmologist will reveal whether shadows are cast on the retina as the light passes through the lens. Shadows result when the protein domains in the lens cast these shadows and indicate a “cloudy” appearance of the lens due to the presence of cataracts. Although this conventional probe allows investigation of the eye's anterior chamber, the lens, and the posterior chamber, such a technique still is non-specific since the shadows cast by protein (e.g., collagen IV, γ-crystallin, or lysozyme, etc.) domains of the same physical size would be identical. Hence having a complementary instrumental technique that is capable of obtaining chemically specific signatures for protein identification and its concentration in the lens and lens capsule, for example, would provide an insight into the specific nature of particle formation in the lens and allow for earlier suitable treatments to be undertaken, before extensive damage has occurred.

For more than 25 years, lensless laser backscatter from fiber optical probes has been used (U.S. Pat. No. 4,776,687) to detect cataracts. Because no lenses were initially used, the proximity of the probe to the eye was uncomfortably close so that the precise scattering volume could be determined. Recently, in order to remove these concerns, a single mode fiber optic backscattering DLS probe was developed (U.S. Pat. No. 5,973,779) to increase the penetration depth of the laser, thereby removing the necessity to bring the probe in close proximity to the eye. Although this new probe now allows investigation of the eye's anterior chamber, the lens, and the posterior chamber, it still is non-specific since scattering by cholesterol, sugar and lysozyme domains of the same size would be the identical. Hence having a complementary instrumental technique capable of obtaining chemically specific signatures for the identification of protein concentration, for example, would provide an insight into the specific nature of particle formation in the lens and allow suitable evasive treatments to be undertaken.

The onset of cataracts is clinically defined as the partial or total opacity of the lens. Much of the research has focused on the lens itself without much attention being placed on the lens capsule, which is also known to undergo changes in thickness, permeability, and elasticity with age.

Over 1.3 million cataract surgeries are performed in the U.S. alone, requiring anywhere from 1-7 days recovery time. Approximately 65,000 of these are less than successful, leaving the patient visually impaired or blind. The development of a noninvasive technique that would provide early treatment (prior to the actual formation of cataracts) or early identification of a predisposition for the development of cataracts is compelling.

Further, detection of glaucoma and retinitis pigmentosa (hereditary disease that causes the rod photoreceptors in the retina to gradually degenerate) is generating interest, but these diseases generally lack diagnostic techniques which can provide advance warning of their onset.

For the medical industry, IR spectroscopy has seen very few clinical applications in the past three decades. However, the interest in using infrared (IR) spectroscopic imaging for disease diagnostics has been growing since the commercial introduction of Fourier Transform IR (FT-IR) imaging systems in the mid-90s. These IR imaging systems detect molecular vibrations and hence do not require the addition of any contrast “agents”. Non-imaging FT-IR instrumentation has been commercially available since 1969 and has been used extensively to study membranes, lung surfactant, protein crystallization, etc., but again only in vitro, since the same instrument limitations as mentioned above for scanning FT-IR apparatus are present.

Diagnosis of the onset of diabetic retinopathy (DR) has for many years been carried out through the use of a conventional opthalmoscope to actually view the retina or through the use of fluorescein angiography, which is, at best, invasive, requiring dye to be injected into the patients arm and spread throughout the body. In the latter, the dye enters the blood stream and then fluorescent images of the retina can be recorded to detect leakage of retinal capillaries, blockages and neovascularization. Although these methods have enjoyed considerable success, they only detect the effects of diabetes after the fact. Having a non-invasive in vivo technique that could detect the onset of DR prior to retinal damage would provide a screening method and could lead to the development of new medical therapies to prevent damage to the retina.

All of the early work has been limited to in vitro studies primarily because of the complex nature of the instrumentation, its scanning mechanism, and the general lack of portability of FT-IR instruments. The main obstacle in bringing IR spectroscopy into the healthcare environment, especially for in vivo applications, is the lack of an easy-to-use instrument and the lack of flexibility in sample positioning. The moving parts in an FT-IR instrument intrinsically limit the portability of an FT-IR instrument, and the stringent optical alignment needed for interferometry further limits the sample position flexibility.

FT-IR spectroscopy has been shown to be useful in differentiating between immature and mature lens capsules through an investigation of changes in protein secondary structure. As the lens ages, there is a change in the concentration of α helical, β-sheet, β-turn and random coil conformation of collagen IV, the primary component of the lens capsule.

In one study, lens capsules removed from 31 cataractous patients (27 had immature cataracts while 4 had mature cataracts) had the FT-IR spectra measured after subtracting the peak intensity of the water band at about 2120-2150 cm⁻¹. Using the band intensities of the amide I (1620-1690 cm⁻¹), amide II (1510-1570 cm⁻¹) and amide III (1240-1340 cm⁻¹) for α helical, β-sheet, β-turn and random coil conformation of collagen IV, changes in the protein structural composition of the lens capsule were correlated with progressive cataract formation.

These results suggested that FT-IR can be used as a diagnostic tool for determining the onset of cataractogenesis. However, for the reasons mentioned above, FT-IR spectroscopy does not lend itself to clinical applications. What is needed is a method and apparatus which allows for in vivo detection of early stage cataractogenesis.

For certain physiological conditions mentioned above, and given an appropriate instrument, IR spectroscopy may be of greater use in revealing new information useful for the advanced detection of disease, i.e., identifying specific disease “markers” or “fingerprints”. What is needed, then, is a portable IR spectrograph with no moving parts, and which is adapted for clinical needs in an outpatient or hospital setting.

In U.S. Pat. No. 6,784,428 by the present inventors and U.S. Pat. No. 6,943,353 by the present inventors and Elmore, various planar array infrared (“PAIR”) spectrographs and methods using IR absorption and no moving parts are disclosed. This apparatus and method are capable of spectral collection in the 3400 to 2000 cm⁻¹ region at high data acquisition rates, primarily by transmission of IR through a sample, i.e., by IR absorption. The instrument is inherently faster and more rugged than the traditional FT-IR instrument and the simple design allows modifications to be easily made for different sample applications. Since the instrument employs a focal plane array (FPA) detector, multiple, independent measurements can be performed simultaneously since the size of the FPA (320×256 pixels) can accommodate up to nine or more spectral images on adjacent pixel rows. As a result, the PAIR spectrograph offers numerous advantages over conventional FT-IR interferometry for a variety of important materials characterization applications. The PAIR technology has demonstrated a sensitivity of 10-100 ppb in less than 30 seconds of data collection time.

Gases, liquid and thin film samples, including molecular monolayers, have been detected successfully with the disclosed PAIR apparatus. Noticeably, the detection of monolayers is known by the FT-IR spectroscopy community as among the most challenging infrared measurements where the system throughput, signal-to-noise ratio, and stability are all pushed to the limits. Further, the PAIR design completely eliminates the need for any moving parts in the system, and therefore a rugged and portable platform can advantageously be built.

FIGS. 3A, 3B, and 3C illustrate conventional PAIR spectrometers that rely upon IR absorption phenomenon and which use no moving parts. However, this conventional device has not been modified for portability suitable for medical diagnosis purposes, particularly for in vivo diagnostic procedures using reflective IR techniques relating to tissue and/or bodily fluids, including eyes, secretions, saliva, and breath, for example. FIG. 5 provides an example of PAIR and FT-IR spectral responses using a polystyrene sample, from which it can be seen that PAIR and FT-IR can provide comparable results over wavenumbers of interest in the IR region.

Although the conventional PAIR system has both high sensitivity and high speed needed for the detection of small concentrations of sample, the 3400-2000 cm⁻¹ nominal spectral range limits the usefulness of the conventional narrow band PAIR technique for protein solution studies. This is due to the limited number of vibrational bands of proteins that have strong absorptions in this region. Although the localized peptide vibrations, amide A and B, and those due to CH stretching are found in the 3400-2900 cm⁻¹ region, the conformationally (α-helix, β sheet, disordered) sensitive IR bands 20 are found in the 1750-800 cm⁻¹ range, and are currently inaccessible using the conventional 3400-2000 cm⁻¹ PAIR instrument.

### SUMMARY

In one embodiment, a method for non-invasively detecting a disease in a patient includes, among other features, providing IR light; reflecting the IR light from a portion of the patient; collecting reflected IR light; dispersing the reflected IR light into a spectrum of reflected IR light; and detecting the spectrum of reflected IR light.

In a further aspect of this embodiment, the method further includes analyzing the spectrum of reflected IR light to identify a molecular fingerprint of the disease.

In another embodiment, an apparatus suitable for non-invasively diagnosing a disease in a patient includes, among other features, an IR light source; light coupling means for coupling at least a portion of the IR light source onto a body part or fluid of the patient and for receiving light reflected from the body part or fluid of the patient; an optically dispersive element arranged in light receiving relation with the light coupling means; and an IR focal plane array which receives dispersed IR light from the optically dispersive element through the light coupling means, wherein the dispersed IR light represents a spectrum of the reflected IR light. Diagnosis of disease in the patient is based, at least in part, on evaluating the spectrum of the reflected IR light, either manually, or by automated means.

In further aspects of this embodiment, the light coupling means may include direct lens coupling, or it may include optical fibers, e.g., a first group of one or more optical fibers which receive light from the IR light source, and a second group of one or more optical fibers arranged to receive reflected IR light from the body part or fluid of the patient. An end portion of the first group of one or more optical fibers located away from the IR light source is suitably arranged facing or touching a body part or fluid of the patient, and an end of the second group of one or more optical fibers located a distance from the body part or fluid of the patient couples the reflected IR light to the optically dispersive element.

In a further aspect of these embodiments, a fiber optic probe head may be used to facilitate the use of the apparatus and method by a clinician for diagnosis of disease in a patient, for example eye disease or diseases which may provide disease markers in the breath, saliva, or other body fluid.

In all embodiments, the apparatus and method are carried out by using no moving parts in the sensor to determine a spectrum and identify a disease marker, except to the extent that a hand-held probe may be involved for a particular application.

### BRIEF DESCRIPTION OF THE DRAWINGS

FIG. 1 depicts an exemplary fiber optic bundle used in an embodiment;

FIG. 2 depicts dual fiber optic bundles used in another aspect of the embodiment of FIG. 4;

FIG. 3A illustrates a conventional PAIR apparatus using IR absorption phenomena;

FIG. 3B illustrates a conventional PAIR apparatus using IR absorption phenomena and multiple sources and samples;

FIG. 3C illustrates a conventional PAIR apparatus using IR absorption phenomena and multiple sources and samples for which respective spectra are spatially separated on the FPA;

FIG. 4 depicts an embodiment which may be used in conjunction with the fiber optic bundles of either FIG. 1 or FIG. 2;

FIG. 5 provides a comparison between PAIR and FT-IR device performance; and

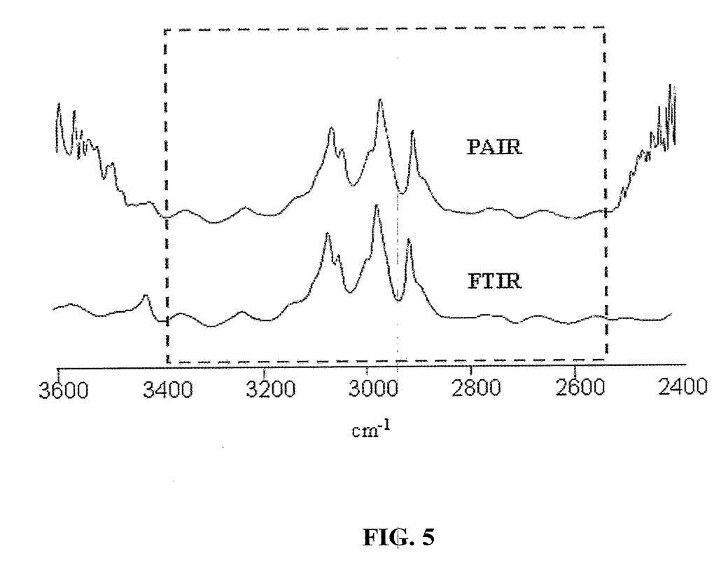

FIG. 6 shows a spectrum of carbon dioxide from human breath.

### DETAILED DESCRIPTION

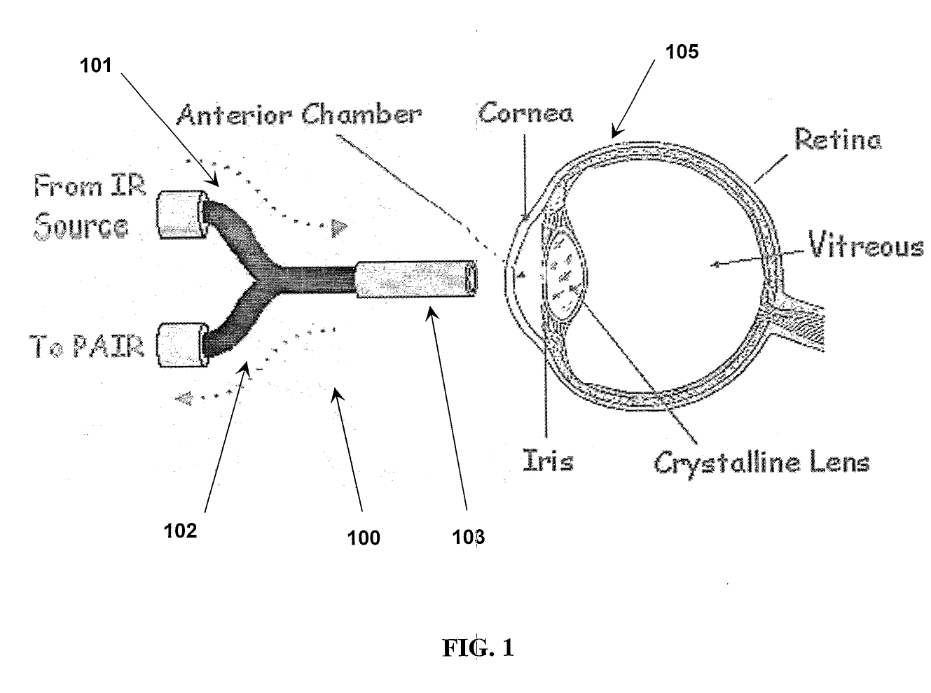

In FIG. 1, IR fiber optic assembly 100 includes an input portion 101 through which an appropriate IR source (not shown) may be coupled to probe head 103. Input portion 101 may include a single optical fiber, or multiple optical fibers. Output portion 102 is also coupled to probe head 103, and may also include a single or multiple fiber optic cables. Including more optical fibers in portions 101 and 102 may result in the achievement of improved light transmission and receiving characteristics. Fibers in portion 101 may be centrally grouped (as viewed in cross-section), and fibers in portion 102 may essentially completely surround central fibers 101. The optical fibers may be mid-IR optical fibers. Chalcogenide optical fibers with losses below 1 dB/m in the mid-infrared range (4000-700 cm⁻¹) have become commercially available in recent years. These multimode fibers offer features such as flexibility and ease-of-use found in their counterparts in the visible and near-IR range. The thermal and mechanical properties of these optical materials have been improved dramatically over the past decade, thus making them suitable for portable and rugged optical devices.

Probe head 103 may simply be a relatively close grouping of fiber ends from fibers I portions 101 and 102, or it may be a more complex fiber optic probe with self-contained optical elements, for example, fiber-optic probe heads such as a Remspec ATR series head (ATR Head HD-01 or Diamond ATR Head HD-11) available through www.remspec.com. These probe heads have conventionally been used with FT-IR apparatus, and with Raman Scattering, a complementary technique to IR spectroscopy, and may include use of an attenuated total reflection (ATR) phenomenon.

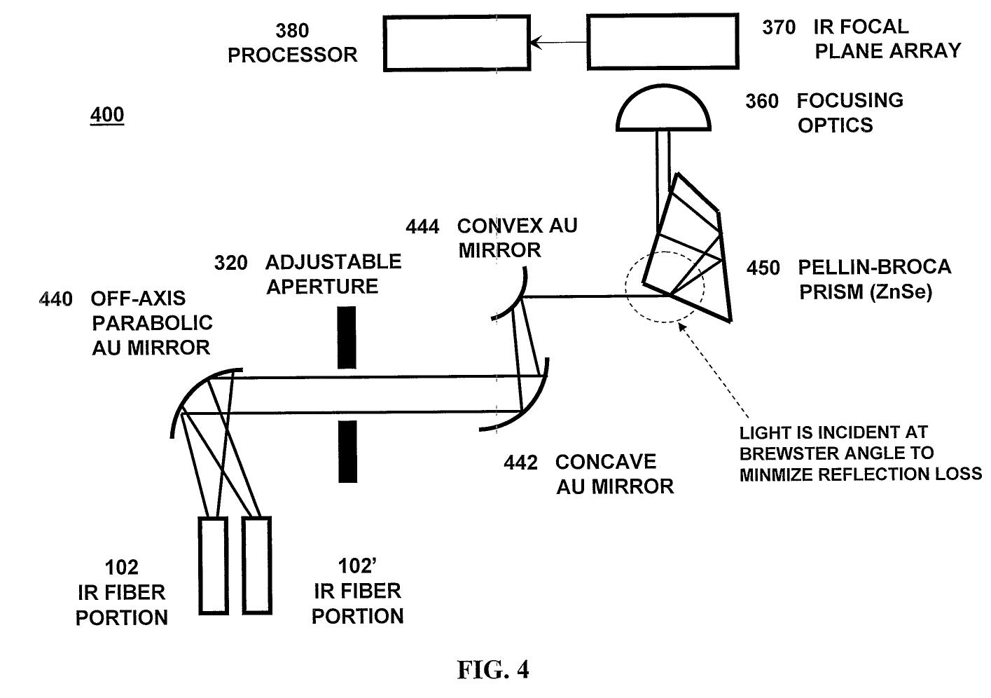

IR light propagating along fibers in portion 101 from the IR source emanates from the end of probe head 103 and may, in one clinical application, be projected or otherwise focused on an eye 105 of a patient. Light reflected from eye 105 is captured by fibers in portion 102, which are also contained in probe head 103. The reflected light captured by fibers in portion 102 may be sent through fiber portion 102 to mirror 440, shown in FIG. 4. Alternatively, IR light may be projected onto a body part or fluid of the patient other than onto an eye. Probe head 103 may be held in proximity to or may contact the body part being examined, and further may be immersed in or otherwise made to contact saliva or may be exposed to exhaled breath of the patient by use of an assembly appropriately configured for interacting IR light with the exhaled breath.

Alternatively, instead of fiber optic bundle 100 and fiber portions 101 and 102, direct lens coupling (not shown) may be used to channel light from the IR source to eye 105 or other tissue/fluid under analysis. In direct lens coupling, the signals are focused into the spectrograph through an aperture. Such conventional non-fiber techniques may be used to capture the light reflected from eye 105, and to further provide an optical path to the modified PAIR system shown in FIG. 4.

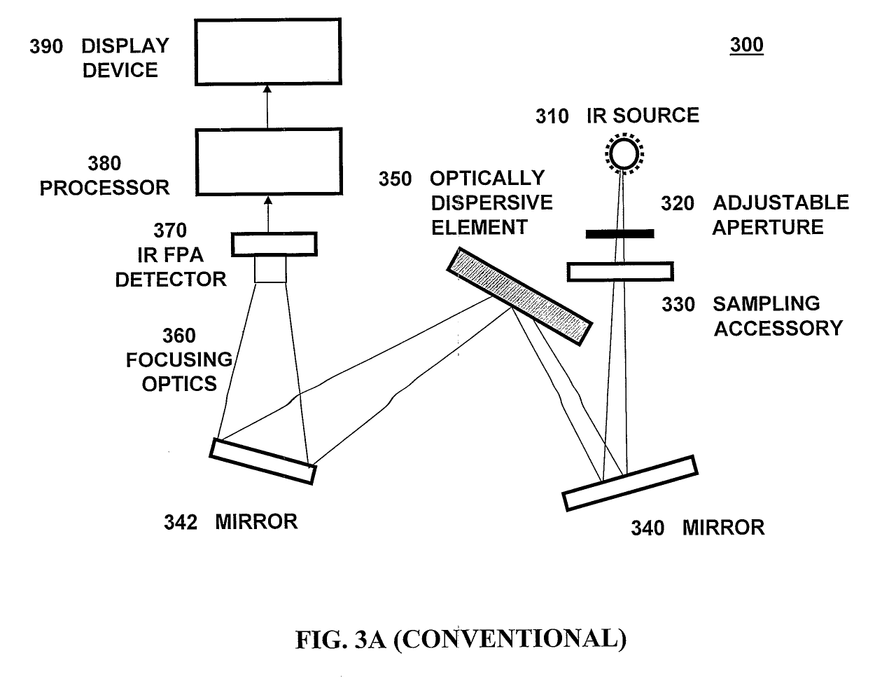

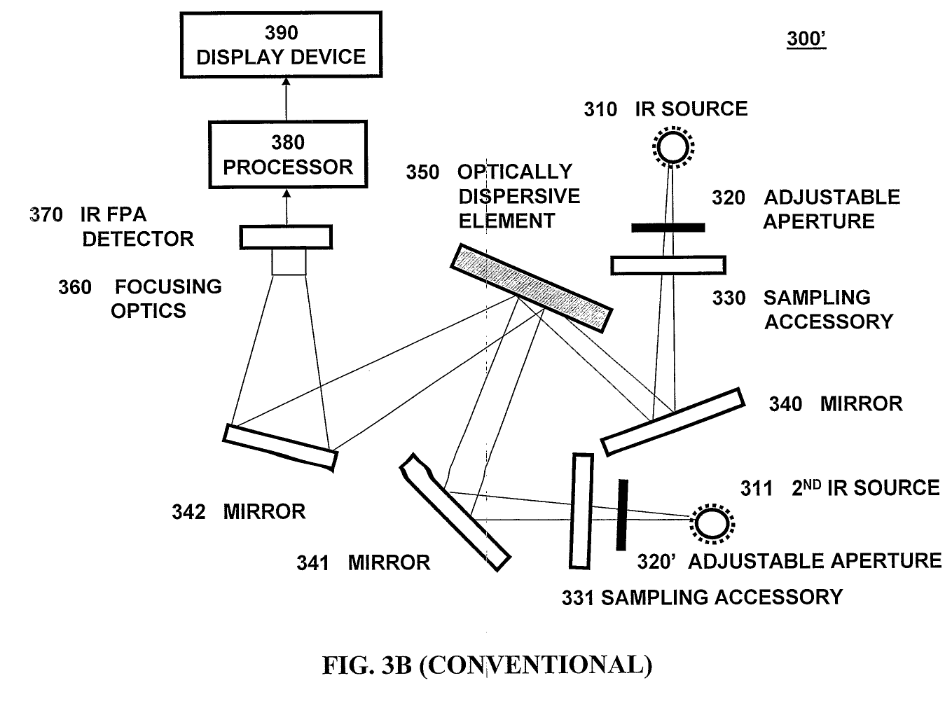

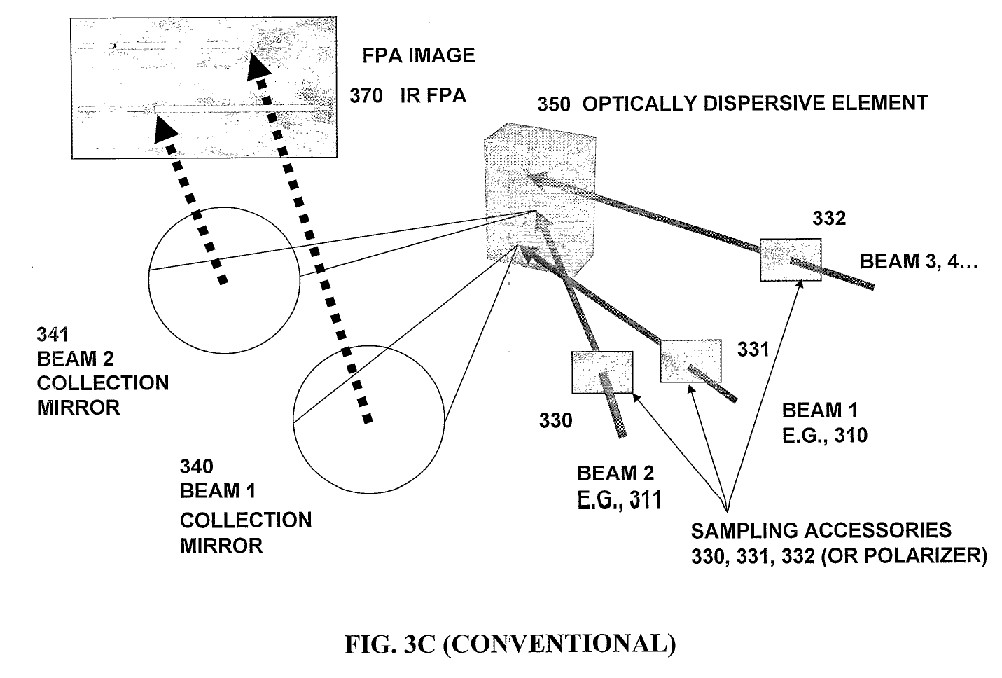

Before further description of the embodiment of FIG. 4, additional background description of a conventional PAIR absorption detector will be provided with reference to FIGS. 3A through 3C.

Apparatus 300 includes an IR light source 310, which may be any common IR light source, including, for example, tungsten lamps, Nernst glowers, glow-bars, or other suitable emission sources. The IR source may be an IR emitter with a ZnSe window or other IR-transparent window. Ideally, IR source 310 has a “flat” or uniform intensity across the IR spectrum, or at least a portion of the IR spectrum. However, if IR source 310 is not uniform, such non-uniformity may be accounted for during an analysis and compensation process.

Adjustable aperture 320 is used, at least in part, to establish the resolution of the apparatus, i.e., a smaller-sized opening provides higher resolution. Adjustable aperture 320 may be an iris or adjustable slit.

Sampling accessory 330 positions the sample volume, which contains a sample to be analyzed, in the optical path. Sampling accessory 320 may be a simple sample holder, which merely positions a small sample volume of material to be sampled, e.g., a polymer film, near the IR source 310, or it may comprise a more elaborate sampling volume arrangement known and used for sampling gases.

Gases, which have a lower density than solids or liquids, may require such a more elaborate sampling accessory having a set of mirrors or other suitable arrangement (not shown) to provide for multiple passes of the IR source through the sample volume. Such multiple passes are useful in ensuring that sufficient optical density is achieved for the IR absorption phenomena to be reasonably measured.

Optically dispersive element 350 receives a portion of an emission from IR light source 310 that is passed through the sample volume. The entire IR spectrum, representative of IR source 310, may not be passed through the sample volume because of the absorption of one or more IR wavelengths in the sample volume within sampling accessory 330. The non-absorbed IR wavelengths then interact with optically dispersive element 350 to form a dispersed light beam, which separates or spreads, in one direction, the wavelengths pre-sent in the IR light exiting sampling accessory 330. Optically dispersive element 350 may be a ruled diffraction grating of a known type, or a prism.

Focusing optics 360 couples light from optically dispersive element 350 into IR detector 370 which has a plurality of detection elements arranged at least along a dispersion direction corresponding to the direction of the dispersed light beam. Typically, incident light is projected onto more than one row of pixels, and the projected light from the optically dispersive element may cover 20 pixels. IR FPA detector 370 detects the dispersed light beam from optically dispersive element 350, and provides an output, which is subsequently used to determine the IR spectral information of the sample in the sample volume contained in sampling accessory 330. Processor 380 analyses the IR FPA data, and display device 390 may provide a visual representation of the sample spectral information.

In FIG. 3B, a second IR source 320′ and related optical components (i.e., adjustable aperture 320′, sampling accessory 331, and mirror 341) have been added, demonstrating the ability of the PAIR technology to “multiplex”, and provide for simultaneous sampling and analysis of multiple samples.

In FIG. 3C, such multiplexing is illustrated as “spatial multiplexing”, i.e., wherein the spectral content of multiple samples are spatially separated on the face of IR FPA 370, allowing simultaneous and independent detection of multiple sample spectra.

Returning to the embodiment of FIG. 4, the IR light source may be in a mid-IR region including wavenumbers in the range of 4000 cm⁻¹ to 400 cm⁻¹, or may be in a far-IR region including wavenumbers in the range of 400 cm⁻¹ to 5 cm⁻¹. The far-IR region of the spectrum contains protein bands characteristic of protein confirmations which are correlated to disease markers. This region has not been exploited for early stage detection of disease.

Apparatus 400 may included an optically dispersive element such as a Pellin-Broca prism 450. In IR wavelengths, the Pellin-Broca prism may be machined from zinc selenide (ZnSe) in order to minimize the material absorption in certain IR spectral ranges, and to ensure adequate optical dispersion as a function of wavelength. A Pellin-Broca prism implementation may be desirable in order to achieve a compact and portable design, given the ability of such a prism to “turn” the light passing through prism 450 by 90 degrees in a relatively small space, as further described below.

Apparatus 400 operates similarly to apparatus 300 shown in FIG. 3A. However, light coupling means may include IR fiber portion 102 which, as described above with respect to FIG. 1, may be a multi-fiber bundle, or may be through direct lens coupling (not shown). Light from IR fiber portion 102 may be provided to off-axis parabolic mirror 440; concave mirror 442; and convex mirror 444 along a known type of optical path. The light being projected by IR fiber portion 102 includes light reflected from a sample being illuminated, for example, eye 105.

By reflecting IR light from a sample or eye 105, certain wavelengths are absorbed by the target, and others are reflected off the target. Both the spectrum of the reflected IR light and the spectrum of the absorbed IR light can provide insight into the chemical composition of the target, as discussed above.

Focusing optics 360 may be a germanium (Ge) condensing lens used to properly project the light emanating from prism 450 onto IR FPA detector 370. The parabolic-shaped mirrors are preferable when using an IR fiber, in order to collimate the cone-shaped fiber output light beam. A ruled diffraction grating may be used with fiber optics, assuming that appropriate measures are taken to collimate the conical beam emanating from the fiber, and to couple the light into the system and onto the diffraction grating.

Although a diffraction grating can provide adequate resolution for many applications, the Pellin-Broca geometry provides at least three benefits: (1) optical dispersion is only a function of the refractive indices at different wavelengths, thus simplifying the optical design; (2) the two-in-one prism design has a very high angular dispersion efficiency, and the approximate 90° beam folding available allows a compact footprint of the optical system to be achieved for a compact, portable and integrated design; and (3) a Brewster angle incident configuration may be utilized in order to maximize the transmission of light at the ambient/ZnSe interface. The latter may be of some importance in the IR range where reflection loss may be a major concern due to the high refractive index of ZnSe (˜2.4).

Besides the Pellin-Broca prism design, special diffractive gratings optimized for mid or far-IR performance, may provide similar, if not better throughput and dispersion than a prism approach. However, the dependence of resolution on both the groove number and grating size may put more constraints on the optical design using gratings. Therefore, the use of gratings may be considered where low-cost off-the-shelf gratings with low groove numbers will suffice for the particular application, and in situations where higher resolution is required than can be obtained with prisms.

In either case of using a prism or a diffraction grating, optically dispersive element 350 may be adjustable with respect to an angle of incidence between its surface and incident light which is projected onto the surface. Such an angular adjustment may be used to control the wavelength range, or spectral bandpass that is presented to IR detector 370.

IR FPA detector 370 may be an InSb camera sensitive in the 3-5 μm wavelength range, for example. InSb detectors in this range may also be thermoelectrically cooled to enhance portability.

IR FPA detector 370 may alternatively be a mercury-cadmium-telluride HgCdTe (MCT) array, which has improved sensitivity and bandwidth in comparison to the InSb device, for example. Using an MCT FPA, “real-time” detection of the chemical “fingerprint” of solid and liquid samples in the 1725-800 cm⁻¹ region are achievable. An instrument that operates in the 1725-800 cm⁻¹ region would allow for the study of collagen IV and γ-crystallin. An MCT focal plane array potentially can cover the region from 4000-800 cm⁻¹. In order to avoid optical constraints by the use of a 128×128 MCT array when the dispersive element is a grating, a narrower band of frequencies (1725-800 cm⁻¹) may be suitable for some diagnostic techniques.

A grating has the advantage of being flexible in terms of its dispersion power, which is easily controlled by the groove density. But for broadband operation, there is a concern with the multiple diffraction orders from a grating. Interfering orders superimposed on the same part of the spectrograph can pose a problem. The use of a prism, however, is simpler in terms of design, but often only limited dispersion power can be achieved.

After the IR focal plane array receives the dispersed IR light from the optically dispersive element, spectral data is analyzed by processor 380, and a diagnosis of disease in the patient is based, at least in part, on the analyzed spectrum of the reflected IR light. Such analysis may be done manually by a clinician, or the diagnosis may be automated by an appropriate software program which is capable of recognizing various disease markers, as discussed.

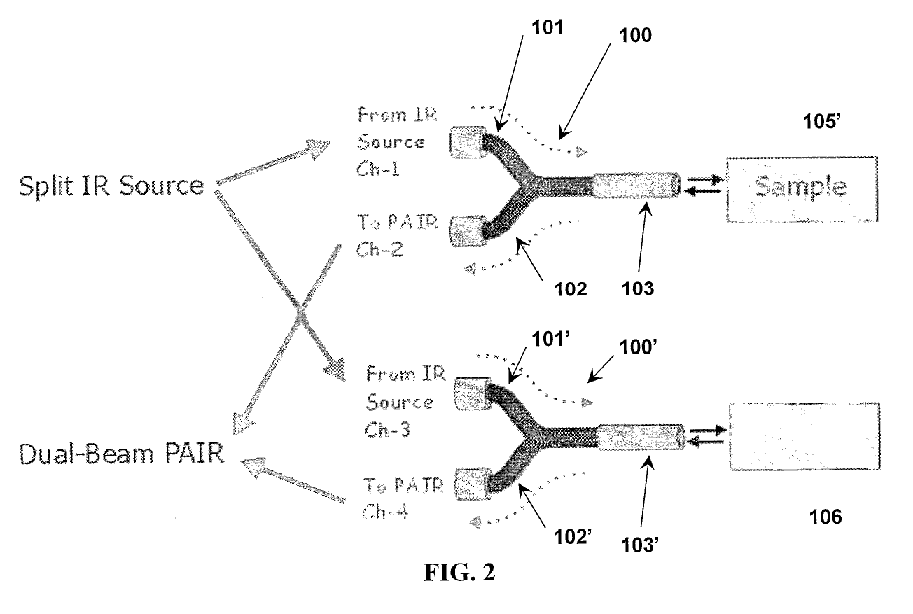

FIG. 2 illustrates an aspect of an embodiment in which compensation of the spectrum of a sample, e.g., the spectrum of light reflected off a body part, is made possible to remove the effects of the environment. For example, water is commonly present in biological material, and water vapor is commonly present in the atmosphere. The eye typically contains a relatively large amount of water, which may undesirably mask the spectral information of various disease markers. In FIG. 2, dual fiber bundles 100 and 100′ are provided. Fiber bundle 100 has been previously described, and eye 105 has been generalized to sample 105′ which could be body tissue, fluid, or exhaled breath, for example. Fiber bundle 100′ is arranged similarly to bundle 100. However, a portion of the IR source may be directed through fiber portion 101′ onto reference 106, and reflected IR light from reference 106 may be received by probe 103′, and directed through fiber portion 102′ to mirror 440 in FIG. 4.

In another aspect of an embodiment, using the multi-channel capability of the PAIR apparatus in FIG. 4 as exemplified by FIGS. 3B and 3C, for example, four signals (or more) may be projected onto IR FPA 370, i.e., signals in fiber portions 101, 102, 101′, and 102′ may be analyzed, given appropriate optical entrance arrangements in FIG. 4 with respect to mirror 440. Such an arrangement allows for simultaneous detection of the spectrum of the reference and the spectrum of reflected IR light. Processor 380 may then correct the spectrum of the sample by known subtractive or ratio techniques. Separate processing of each of multiple signals is made possible by projecting optically dispersed light onto different spatial areas of IR FPA 370.

In another embodiment, a method for non-invasively detecting a disease in a patient includes providing IR light which is reflected from a portion of the patient. Reflected IR light from the patient is collected, and then provided to an optically dispersive element which disperses the reflected IR light into a spectrum of reflected IR light. The dispersed light is projected onto a focal plane array and detected. Thereafter, the spectral information is analyzed to identify a molecular fingerprint of a disease.

In an aspect of the method, IR light is reflected from an eye of the patient, and the analysis of the spectrum of reflected IR light provides the ability to diagnose an eye disease, including an early stage of cataractogenesis, diabetic retinopathy, glaucoma, or retinitis pigmentosa in an eye of the patient.

In another aspect of the method, reflecting IR light from an eye of the patient may be used to non-invasively characterize ocular fluid in the eye of the patient to identify one or more proteins contained therein which may be indicative of a disease precursor or marker. The IR light may be coupled through a first group of one or more optical fibers and reflected IR light may be collected with a second group of one or more optical fibers.

In a further aspect of the method, a probe head may be coupled to an end of the first group of one or more optical fibers and an end of the second group of one or more optical fibers. The probe head may be placed in contact with or in proximity to a body fluid, e.g., saliva or exhaled breath (liquid or gas), or a body tissue of the patient. The reflected IR light may then be collected through the probe head.

In addition, a spectrum of a reference and the spectrum of reflected IR light from an aqueous sample, e.g., fluid in the eye, may be simultaneously collected so that the spectral information relating to the patient may be compensated. A reference may comprise water or water vapor, for example, since water is prevalent in biological material, and may otherwise act to mask disease markers or fingerprints.

Depending on particular diagnostic needs, IR light may be provided in a mid-IR region including wavenumbers in the range of 4000 cm⁻¹ to 400 cm⁻¹ or in a far-IR region including wavenumbers in the range of 400 cm⁻¹ to 5 cm⁻¹. IR spectrographic analysis in each of these ranges may provide complementary analytical information.

The use of an IR fiber optic diamond coated ATR probe coupled to a portable broad band PAIR instrument described above makes it possible to detect certain chemical/biological components in saliva. One way to do this is to touch the tongue with the diamond ATR probe lightly or instead “swab” saliva from the tongue and place in on the ATR probe. Using diamond coatings or bulk diamond ATR crystals will allow for easy sterilization and re-use.

For example, PAIR with a fiber optic probe could be implemented in the treatment of endometriosis in women where it is critically important to assess the amount of bioavailable progesterone in the body when prescribing supplemental topical levels of progesterone. One of the issues with the current “blood test” methods for determining progesterone concentration is that they detect the serum concentration of progesterone (that which is thought to be protein bound) and not the amount of lipophilic progesterone that is taken up gradually by red blood cell membranes after topical application to the skin. Since the progesterone transported by red blood cell membranes is readily available to all target tissues and to saliva, in vivo PAIR protocols for measuring the concentration of progesterone in saliva is achievable. Because the chemical “fingerprint” of progesterone is unique, it will be detectable in the presence of the multiple other components found in saliva and, after calibration, the intensity of the IR peaks can be used to quantitatively determine the amount of progesterone present.

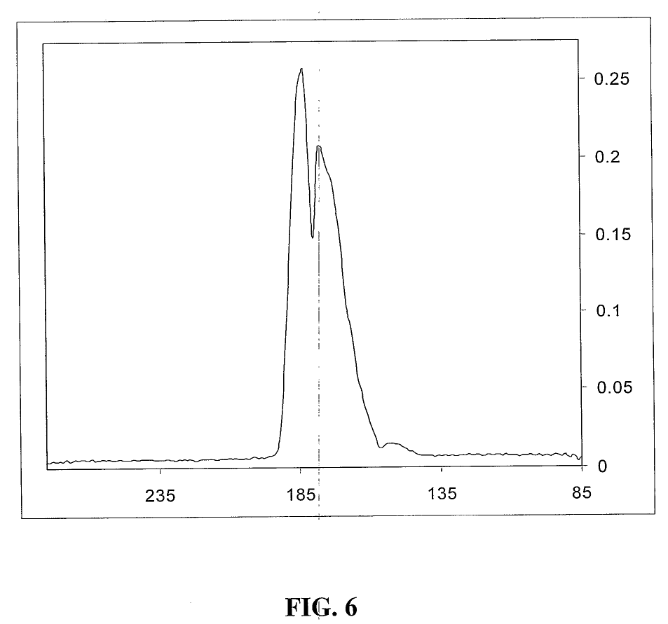

In another aspect of the disclosure, and with reference to FIG. 6, the spectrum of carbon dioxide from human breath is shown. A normal person usually breathes out between 1 to 1.5% of CO₂. At 1.5 ms total integration time, the signal level is at 0.25 absorbance units, while the noise of the PAIR is about 2.7×10⁻³ for a single-frame, single-row collection. This gives a SNR of about 100. On the other hand, if a combination of row binning and frame averaging is used, one can obtain a noise level of 2.2×10⁻⁴ in 0.5 seconds, giving an SNR of nearly 1000. This capability places PAIR's gas sensitivity in the sub-mg/m³, or ng/cm³ level, or at about 0.001%. At this level of sensitivity, volatile organic compounds (VOC) that have been associated with a number of medical conditions as indicated in Table I below, and which can be detected by the apparatus and method of this disclosure.

TABLE I

| Disease | Source | Identity of VOC |
|---|---|---|
| Breast cancer | human breath, | 2,3-dimethyl-pentane, |
| | lung air | 2-methyl-pentane, |
| | | 3-methyl-pentane |
| Lung cancer | human breath, | alkanes, mono-methylated |
| | lung air | alkanes, aniline, |
| | | o-toluidine |
| Acute asthma | human breath | pentane |
| Rheumatoid | alveolar air | pentane |
| arthritis | | |
| Cardiopulmonary | alveolar air | acetone, ethanol |
| disease | | |
| Uremia | breath, urine | dimethylamine, trimethylamine |
| Larynx cancer | breath | C₂ to C₆ aliphatic acids |
| Cirrhosis | breath | acetic acid, propionic acid, |
| | | isobutyric acid, butyric acid, |
| | | isovaleric acid, carbon disulphide |

Further, in another application, the above method and apparatus allows the detection of airborne viruses and bacteria in hospital environments. Due to its extreme sensitivity (100-1000× more sensitive than FT-IR) the broad band PAIR instrument disclosed in its various embodiments and aspects can identify the presence of small concentrations (ppb or less) of bacterial or viral contaminants in the air.

Further, the engineering process for miniaturizing any optical instrumentation shares some common requirements including limitations to reduction of dimensions by physical laws, use of smaller components which maintain adequate performance, and shorter travel length for the moving parts, if any.

For the novel diagnostic instrument of this disclosure, the miniaturization process faced the challenges posted by the first two of the three requirements. Both the availability of smaller components and the reduction of the required optical paths must be satisfied before effective miniaturization of the new PAIR instrument can be accomplished. On the other hand, due to the no-moving-parts design, there are no constraints due to the travel length requirement and the space needed for accommodating the servo or control components.

IR radiation, when compared with visible or ultraviolet light, has wavelengths 10 to 100 times longer. As a result, the diffraction and refraction of the IR radiation tends to follow vastly different, usually longer, geometrical paths than that of ultraviolet (US) and visible light. Minimizing the overall footprint of a PAIR instrument is, therefore, more difficult from the design point of view. On the other hand, once a compact design of the PAIR is implemented, the higher tolerances at these longer wavelengths (5-12 μm) will prevent beam misalignment, thus making the PAIR instrument more rugged. Due to the no-moving-parts design, the PAIR is more stable against any mechanical or thermal drift.

In terms of the components required for miniaturization, the availability of the smaller IR optics and devices needs to be taken into consideration. For example, operation temperature of an MCT array is usually at 77 K, or the liquid nitrogen temperature. This means that a cooling mechanism must be used in order for the detector to function properly. A liquid nitrogen (LN₂) dewar with a cold-finger in contact with the FPA is commonly used for this purpose. However, the size of the dewar and the required vertical orientation put limitations on the miniaturization process. To this end, a closed-cycle cryo cooler (Stirling Cooler) (not shown) may be used to operate the MCT array at 60 to 80 K. For a 512 by 512 MCT array, a 4 W Stirling cooler approximately the size of a coffee mug provides the necessary heat dissipation. Alternatively, thermo-electrically (TE) cooled detectors may be used to aid in miniaturization and portability. Further, additional materials sensitive to radiation in the far-IR region are continuing to be developed into detectors including focal plane arrays, for example, GaAs and Ge.

One challenging task involved in designing the portable spectrograph was resizing and redirecting the mid-infrared beam from the entrance in FIG. 4 so that it passes through the dispersive medium in a highly collimated fashion, and is eventually focused onto IR FPA 370 as a finely resolved spectroscopic line image. State of the art optical design software (OSLO Premium, Lambda Research Corporation, Littleton, Mass.) facilitated the ability to model and optimize the detailed optical performance of different designs before a prototype was built. Another issue is the specifications of the IR arrays. The use of FPAs with either a Sterling cooler, those that use compact LN₂ dewar for cooling, or those that are TE-cooled may be used and are commercially available.

The above disclosure allows a multicomponent analysis to be carried out simultaneously and, when applied to the field of eye diagnostics, for example, diabetic retinopathy, cataractogenesis, etc., it can provide an “early warning” diagnosis since the apparatus and method have sensitivities to parts per billion (molecular concentrations) which is achievable with the above-described broadband PAIR instrument and method.

### STATEMENT OF INDUSTRIAL APPLICABILITY

This disclosure has application to the medical field, and particularly has applicability to medical diagnosis of disease.

What is claimed as new and desired to be protected by Letters Patent of the United States is:

1. A method for non-invasively detecting a disease in a patient, comprising:
providing IR light;
reflecting the IR light from a portion of the patient;
collecting reflected IR light;
dispersing the reflected IR light into a spectrum of reflected IR light; and
detecting the spectrum of reflected IR light.

2. The method of claim 1, further comprising analyzing the spectrum of reflected IR light to identify a molecular fingerprint of the disease.

3. The method of claim 2, further comprising reflecting the IR light from an eye of the patient, wherein the analysis of the spectrum of reflected IR light is capable of diagnosing an eye disease.

4. The method of claim 3, wherein the analysis of the spectrum of reflected IR light is capable of diagnosing an early stage of cataractogenesis in an eye lens of the patient.

5. The method of claim 3, wherein the analysis of the spectrum of reflected IR light provides a diagnosis of diabetic retinopathy.

6. The method of claim 3, wherein the analysis of the spectrum of reflected IR light provides a diagnosis of glaucoma in an eye of the patient.

7. The method of claim 3, wherein the analysis of the spectrum of reflected IR light provides a diagnosis of retinitis pigmentosa in an eye of the patient.

8. The method of claim 1, further comprising reflecting the IR light from an eye of the patient and non-invasively characterizing ocular fluid in the eye of the patient and identifying one or more proteins contained therein.

9. The method of claim 1, further comprising coupling the IR light through a first group of one or more optical fibers and collecting reflected IR light with a second group of one or more optical fibers.

10. The method of claim 9, further comprising:
coupling a probe head to an end of the first group of one or more optical fibers and an end of the second group of one or more optical fibers;
placing the probe head in contact with at least one of a body fluid and a body tissue of the patient; and
collecting the reflected IR light through the probe head.

11. The method of claim 10, wherein the probe head is placed in proximity to a body fluid comprising a gas.

12. The method of claim 1, further comprising simultaneously detecting a spectrum of a reference and the spectrum of reflected IR light.

13. The method of claim 12, further comprising compensating the spectrum of reflected IR light by using the reference spectrum.

14. The method of claim 12, wherein the reference is water.

15. The method of claim 12, wherein the reference comprises water vapor.

16. The method of claim 1, further comprising providing IR light in a mid-IR region including wavenumbers in the range of 4000 cm⁻¹ to 400 cm⁻¹.

17. The method of claim 1, further comprising providing IR light in a far-IR region including wavenumbers in the range of 400 cm⁻¹ to 5 cm⁻¹.

18. An apparatus suitable for non-invasively diagnosing a disease in a patient, the apparatus comprising:
an IR light source;
light coupling means for coupling at least a portion of the IR light source onto a body part or fluid of the patient and for receiving light reflected from the body part or fluid of the patient;
an optically dispersive element arranged in light receiving relation with the light coupling means; and
an IR focal plane array which receives dispersed IR light from the optically dispersive element through the light coupling means,
wherein the dispersed IR light represents a spectrum of the reflected IR light, and
wherein the diagnosis of disease in the patient is based, at least in part, on the spectrum of the reflected IR light.

19. The apparatus of claim 18, wherein the light coupling means comprises:
a first group of one or more optical fibers in light receiving relation to the IR light source and a second group of one or more optical fibers arranged to receive reflected IR light from the body part or fluid of the patient,
wherein an end portion of the first group of one or more optical fibers distal from the IR light source is suitably arranged facing a body part or fluid of the patient, and
wherein an end of the second group of one or more optical fibers distal from the body part or fluid of the patient couples the reflected IR light to the optically dispersive element.

20. The apparatus of claim 18, wherein the first and second groups each comprise multiple fibers arranged in a bundle, wherein the first group is essentially surrounded by the second group.

21. The apparatus of claim 18, further comprising a probe head operatively arranged with respect to the light coupling means, wherein the probe head is placed in contact with the body part or fluid of the patient.

22. The apparatus of claim 19, further comprising a probe head coupled to an end portion of the first group of one or more optical fibers opposed to the body part or fluid of the patient,
wherein the probe head is placed in contact with the body part or fluid of the patient, and
wherein the probe head is coupled to the second group of one or more optical fibers containing IR light reflected from the body part or fluid of the patient.

23. The apparatus of claim 21, wherein the probe head is configured as an attenuated total reflection probe.

24. The apparatus of claim 18, further comprising means for simultaneously detecting a spectrum of a reference and the spectrum of reflected IR light.

25. The apparatus of claim 24, further comprising means for compensating the spectrum of reflected IR light by using the reference spectrum.

26. The apparatus of claim 24, wherein the reference is water.

27. The apparatus of claim 24, wherein the reference comprises water vapor.

28. The apparatus of claim 24, wherein the means for simultaneously detecting comprises means for projecting the spectrum of the reference and the spectrum of reflected IR light on different areas of the IR focal plane array.

29. The apparatus of claim 18, wherein the IR focal plane array is thermo-electrically cooled.

30. The apparatus of claim 18, wherein the optically dispersive element comprises a prism.

31. The apparatus of claim 30, wherein the prism comprises a Pellin-Broca prism.

32. The apparatus of claim 18, wherein the IR focal plane array is sensitive in a mid-IR region including wavenumbers in the range of 4000 cm⁻¹ to 400 cm⁻¹.

33. The apparatus of claim 18, wherein the IR focal plane array is sensitive in a far-IR region including wavenumbers in the range of 400 cm⁻¹ to 5 cm⁻¹.
